(19) United States
(12) Patent Application Publication
(10) Pub. No.: US 2024/0198001 A1
(43) Pub. Date: Jun. 20, 2024

(54) NEEDLE-FREE INJECTION SYSTEMS

(71) Applicant: ZOETIS SERVICES LLC, Parsippany, NJ (US)

(72) Inventors: STEVEN CHARLES DEANE, CAMBRIDGE (GB); ANDREW GARETH FORBES, CAMBRIDGE (GB); WAYNE JOHN GIMBLETT, CAMBRIDGE (GB); CHARLOTTE LOUISE GISBOURNE, CAMBRIDGE (GB)

(21) Appl. No.: 18/540,959

(22) Filed: Dec. 15, 2023

Related U.S. Application Data
(60) Provisional application No. 63/476,177, filed on Dec. 20, 2022.

(51) Int. Cl. A61M 5/20; A61M 5/30; A61M 5/315; A61M 39/22

(52) U.S. Cl. CPC A61M 5/2046; A61M 5/30; A61M 5/31511; A61M 39/22

(57) ABSTRACT

A gas-actuated needle-free injector is provided. Such gas-actuated needle-free injector includes a gas spring in communication with a piston. A vaccine dosing chamber is configured to receive one or more vaccines or components thereof. A ball screw is coaxial with the gas spring and the vaccine dosing chamber and configured to receive at least portion of the piston. The ball screw is configured to move the piston between a first position and a second position. Associated systems and methods are also provided.

### CROSS REFERENCE TO RELATED APPLICATION

This application claims the benefit of U.S. Provisional Patent Application No. 63/476,177, filed Dec. 20, 2022, which is expressly incorporated herein by reference in its entirety.

### FIELD

The present disclosure relates to needle-free injection systems, and more specifically, to needle-free injection systems including actuated gas springs for pressure regulation, axial push back mechanisms, and/or electrical hydraulic triggers.

### BACKGROUND

This section provides background information related to the present disclosure which is not necessarily prior art.

Needle-containing injectors have traditionally been used to inject medications and the like for various animal and human health applications. These apparatuses, however, have serious drawbacks, including, for example, increased risk of disease transmission among injected recipients, as well as breakage and possible tissue damage at injection sites, which pose serious health concerns, and also, contribute to significant profit losses for meat producers. Needle-free injection systems address many of these concerns. Mechanical springs are often used to provide compact energy storage mechanisms for needle-free injections. Force adjustments, however, are often needed to vary injection depths, for example, between day old piglets to grown sows. Such adjustments are often impractical with mechanical springs. One common alternative includes pneumatically driven systems. These systems, however, often require tethering of injection systems to compressors, which impacts ergonomics. Gas springs are an alternative to mechanical and pneumatically driven systems and are often considered as giving the lowest mass spring for needle free injection. Gas springs, however, often undesirably drift with temperature (e.g., variations of ambient temperature and/or cooling following gas compression, for example, during priming). Further, mechanical frames are often required to prevent twisting and accelerated seal wear for both mechanical and gas springs. Such mechanical frames can undesirably increase overall mass of needle-free injection systems. Likewise, mechanical trigger mechanisms are often used to release gas or mechanical springs. Such mechanisms, however, often experience associated wear and lifetime issues because of the large amounts of applied force. One common alternative includes mechanically operated hydraulic triggers, which require the pressure in hydraulic hoses to drop for reset, limiting the ability to deliver small doses accurately. Accordingly, it would be desirable to develop apparatuses and methods for addressing each of these concerns.

### DRAWINGS

The drawings described herein are for illustrative purposes only of selected embodiments and not all possible implementations, and are not intended to limit the scope of the present disclosure.

FIG. 1 is a perspective, cross-sectional illustration of an example gas-actuated needle-free injector in accordance with various aspects of the present disclosure, where a gas spring is fully extended;

FIG. 2 is another perspective, cross-sectional illustration of the example gas-actuated needle-free injector in accordance with various aspects of the present disclosure, where the gas spring is fully extended;

FIG. 3 is a close-up illustration of a receiving port of a gas-actuated needle-free injector in accordance with various aspects of the present disclosure;

FIG. 4 is a cross-sectional illustration of a nozzle end of a vaccine dosing chamber of the example gas-actuated needle-free injector in accordance with various aspects of the present disclosure;

FIG. 5 is another cross-sectional illustration of a nozzle end of a vaccine dosing chamber of the example gas-actuated needle-free injector, with a solenoid actuator in an “off” position, in accordance with various aspects of the present disclosure;

FIG. 6 is another cross-sectional illustration of a nozzle end of a vaccine dosing chamber of the example gas-actuated needle-free injector, with a solenoid actuator in an “on” position, in accordance with various aspects of the present disclosure;

FIG. 7 is a cross-sectional illustration of an example gas-actuated needle-free injector in accordance with various aspects of the present disclosure;

FIG. 8 is a schematic illustration of the vaccine dosing chamber of the example gas-actuated needle-free injector in accordance with various aspects of the present disclosure;

FIG. 9 is an enlarged illustration of the receiving port of the vaccine dosing chamber of FIG. 8;

FIG. 10 is a close-up illustration of a motor-belt system of the gas-actuated needle-free injector in accordance with various aspects of the present disclosure;

FIG. 11 is a partial cross-sectional illustration of the example gas-actuated needle-free injector in accordance with various aspects of the present disclosure, where the gas spring is fully extended;

FIG. 12 is a partial cross-sectional illustration of the example gas-actuated needle-free injector in accordance with various aspects of the present disclosure, where a piston of the gas spring is pushed back by a ball screw, the action of which causes a vaccine(s) and/or vaccine components to be drawn into a vaccine dosing chamber;

FIG. 13 is a partial cross-sectional illustration of the example gas-actuated needle-free injector in accordance with various aspects of the present disclosure, where the gas-actuated needle-free injector is in a loaded state ready to administer the vaccine(s) and/or vaccine components;

FIG. 14 is a partial cross-sectional illustration of the example gas-actuated needle-free injector in accordance with various aspects of the present disclosure, where a hydraulic valve is in an open position to administer the vaccine(s) and/or vaccine components and administration continues until the piston reaches an endstop;

FIG. 15 is a partial cross-sectional illustration of the example gas-actuated needle-free injector in accordance with various aspects of the present disclosure, where a piston of the gas spring is in a fully extended position;

FIG. 16 is a partial cross-sectional illustration of the example gas-actuated needle-free injector in accordance with various aspects of the present disclosure, where a piston of the gas spring is in a retracted position;

FIG. 17 is a partial cross-sectional illustration of the example gas-actuated needle-free injector in accordance with various aspects of the present disclosure, where a piston of the gas spring is in a fully extended position;

FIG. 18 is a partial cross-sectional illustration of the example gas-actuated needle-free injector in accordance with various aspects of the present disclosure, where a piston of the gas spring is in a retracted position; and

FIG. 19 is a schematic of another example gas-actuated needle-free injector in accordance with various aspects of the present disclosure, where a gas spring is fully extended.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

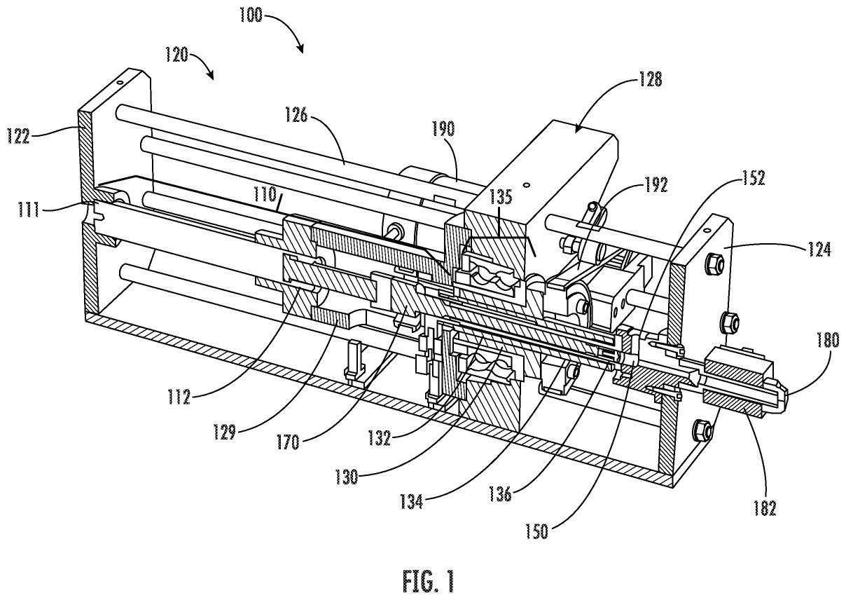

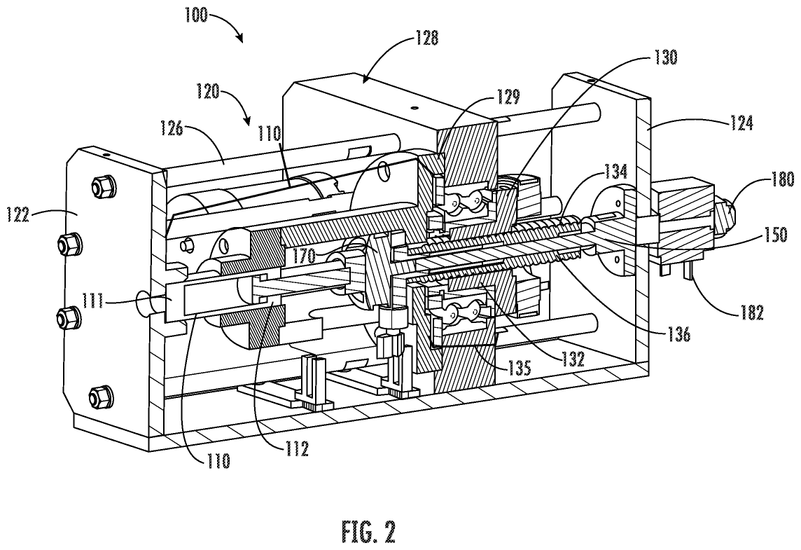

FIGS. 1 and 2 are a cross-sectional illustrations of an example gas-actuated needle-free injector 100. The gas-actuated needle-free injector 100 includes a pneumatic energy storage element (e.g., a gas spring 110) in communication with a dosing chamber (e.g., a vaccine dosing chamber 150), which is a high-pressure chamber configured to receive fluids such as, for example, one or more vaccines and/or components for making a vaccine or vaccines. For example, as illustrated, a piston 170 (e.g., high-pressure piston) may connect the gas spring 110 and the vaccine dosing chamber 150. A housing 120 may surround and support the gas spring 110 and the vaccine dosing chamber 150. For example, the housing 120 may include a first end plate 122 parallel with a second end plate 124 and a plurality of rods (or dowels) 126 extending between and coupled to the first and second end plates 122, 124. The housing 120 may also include a support structure 128 coupled to one or more of the rods 126. As illustrated, the support structure 128 may include, or act as, a linear actuator, such as a ball screw actuator having a ball screw bearing 135. The support structure may further include a motor mount (not shown).

The ball screw bearing 135, vaccine dosing chamber 150, and the gas spring 110 may be coaxial. For example, in certain variations, the support structure 128 may be disposed at an interface between the gas spring 110 and the vaccine dosing chamber 150 to support and align the gas spring 110 and the vaccine dosing chamber 150. As illustrated, a first end 111 of the gas spring 110 may be coupled the first end plate 122, while a second end 112 of the gas spring 110 may be coupled to the support structure 128, and the vaccine dosing chamber 150 may be coupled to the second end plate 124 and extend through the support structure 128 towards the second end 112 of the gas spring 110. As illustrated, in certain variations, the support structure 128 may include one or more couplers 129 that are configured to connect with the second end of the gas spring 112. In certain variations, the vaccine dosing chamber 150 may interface with or extend into the second end 112 of the gas spring 110. For example, the support structure 128 may include a hollow drive nut 130 that defines a cavity 132 that is configured to receive a ball screw 134 having a cavity 136 that receives the piston 170 and is configured to move over the vaccine dosing chamber 150. The piston 170 may also be configured to move through the one or more couplers 129 connecting the support structure 128 and the gas spring 112.

The vaccine dosing chamber 150 is in communication with a nozzle 180 that extends from, or through, the second end plate 124. In certain variations, the nozzle 180 may be formed of a hard-wearing material, such as stainless steel or ruby, and may be removable for cleaning and/or replacement. Further, the nozzle may have a single injection orifice. In comparison, a spray nozzle commonly has multiple small holes pointing in diverse directions. The single injection orifice in accordance with the current disclosure may have an internal diameter greater than or equal to about 200 micrometers (μm) to less than or equal to about 400 μm, and in certain aspects, optionally about 300 μm. In each instance, the nozzle 180 may be contacted to a subject (e.g., sows, piglets) to administer the one or more vaccines and/or components for making a vaccine or vaccines. For example, the injection orifice may be configured to produce a single, coherent jet that penetrates the tissues of the subject.

One or more sensors 140, such as optical switches, may be used to determine positioning of the piston 170. In some instances, for example, a sensed element 145 may be coupled to the piston 170 such that the sensed element 145 interacts with the sensors 140 to determine a position of the piston 170.

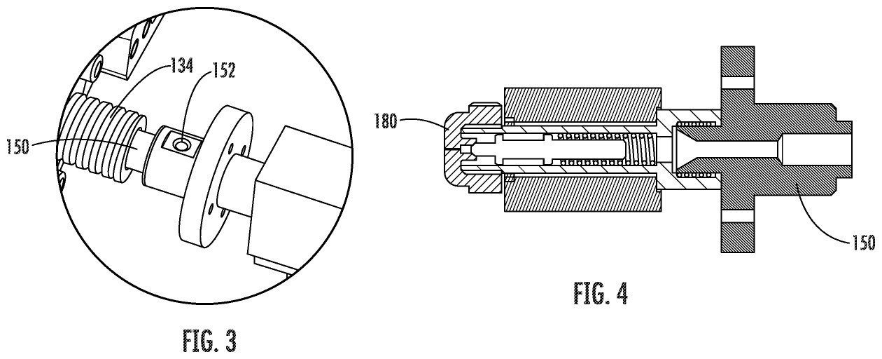

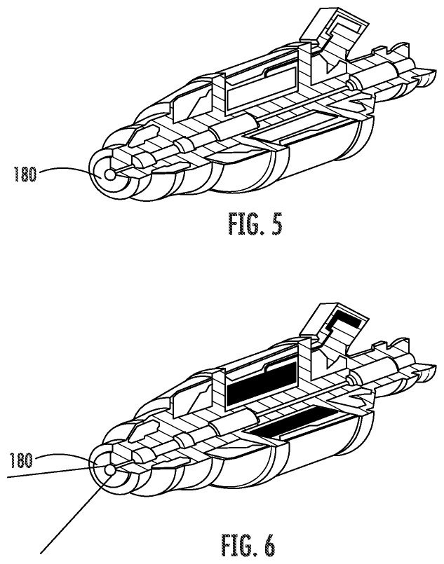

The nozzle 180 includes one or more triggers 182 that are configured to initiate movement and cause the nozzle 180 (and the gas-actuated needle-free injector 100) to move from a closed position to an open position. The one or more triggers 182 may be configured to prevent the movement of air into the nozzle 180. For example, FIGS. 3-6 are cross-section illustrations of the nozzle end of the gas-actuated needle-free injector 100 and, as illustrated, the one or more triggers 182 may actuate a solenoid actuator 300. Such a nozzle 180 may include an injection orifice 302, a valve spring 304, a plunger 306, a casing 308, a filter 310 and an electrical attachment portion 312. FIG. 5 illustrates the solenoid actuator in an “off” position, preventing dispersion of a fluid from the injection orifice 302. FIG. 6 illustrates the solenoid actuator in an “on” position, allowing dispersion of a fluid from the injection orifice 302.

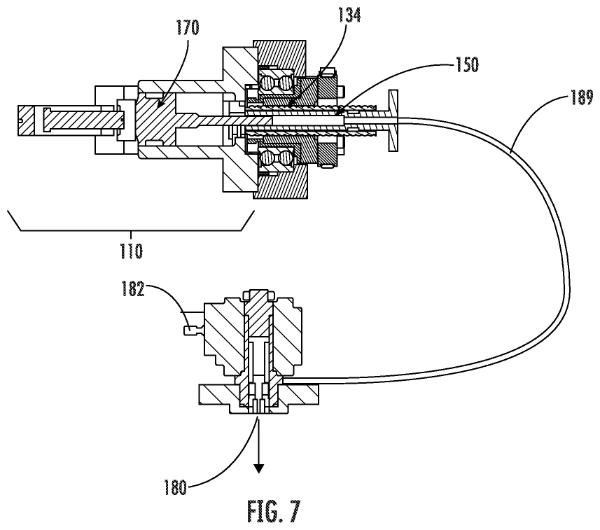

In certain variations, as illustrated in FIG. 7, the vaccine dosing chamber 150 and the nozzle 180 may be connected using a flexible hose 189. The flexible hose 189 may be connected to a wearable handset. In some instances, a pressure hydraulic hose may join the vaccine dosing chamber 150 to the solenoid actuator 300.

Continuing with reference to FIG. 7, according to one aspect, the gas spring 110 may be connected to a dosing chamber 150, which in some instances is under high pressure. This link may be arranged to “float” to allow for tolerance in axial alignment of the gas spring 110 and dosing chamber 150, but with no gap forming as the gas spring 110 is under constant pressure against the piston 170. The dosing chamber 150 may be filled and the gas spring 110 pushed back by a ball screw actuator coaxial with the dosing chamber 150 and the gas spring 110. The dosing chamber 150 may fill by a one-way inlet valve, and a hydraulic trigger valve 160 is closed to prevent air from entering the nozzle. Driving the ball screw actuator back to different positions allows the dose delivered to be varied. When the system is primed, the ball screw actuator retracts, and the position is maintained by the pressure in the hydraulic fluid, which in some instances is a vaccine. To release the injection, an electrically or mechanically operated hydraulic trigger valve 160 is opened, which may be located in a wearable portion or in a handpiece held by an operator. In some instances, the hydraulic trigger valve 160 may form the injection nozzle of the handpiece. The injection finishes either when the piston 170 hits its end stop, or when the hydraulic trigger valve is closed. The latter allowing for more accurate delivery of small doses, as well as allowing for a dose to be varied for a fixed priming position.

In some instances, the hydraulic trigger valve may be an electrical hydraulic trigger, which is non-wearing and does not apply unbalanced forces to the main force generator. Further, more accurate dose control may be achieved for small dosing (e.g., microdoses) since the injection can be shut off electrically, rather than requiring the flexible hose to be depressurized. This is advantageous over a mechanically operated hydraulic trigger, which requires the pressure in the hydraulic hoses to drop for it to reset, thereby limiting the ability to deliver small doses accurately.

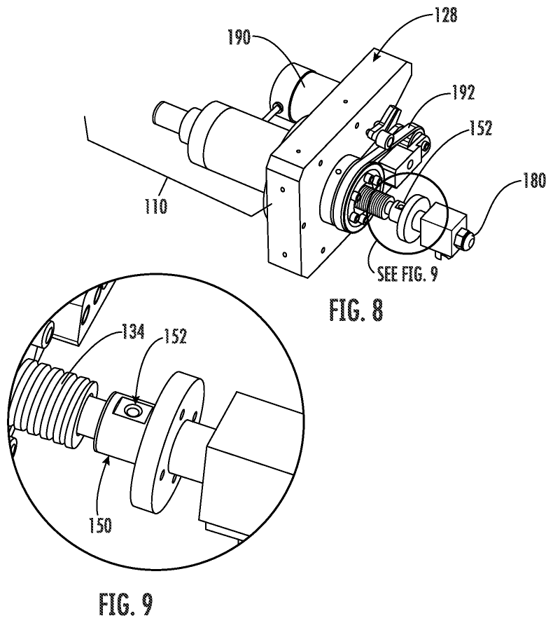

The vaccine dosing chamber 150 may also include one or more one-way entry (or receiving) ports (or openings) configured to receive the one or more vaccines and/or components for making the vaccine. For example, as illustrated in FIGS. 1-4, the vaccine dosing chamber 150 may have a receiving port 152 disposed near the second end plate 124. The receiving port 152 may include a valve configured to communicate with a vaccine vial. For example, in certain variations, the valve may include a plurality of threads with or without an o-ring. In certain variations, the valve may be actuated at low pressures by pushing down from a higher (or top) position, but compresses down to a metal on metal seal at high pressures (e.g., about 600 Bar). FIGS. 3 and 9 provide close-up views of the receiving port 152 in certain instances.

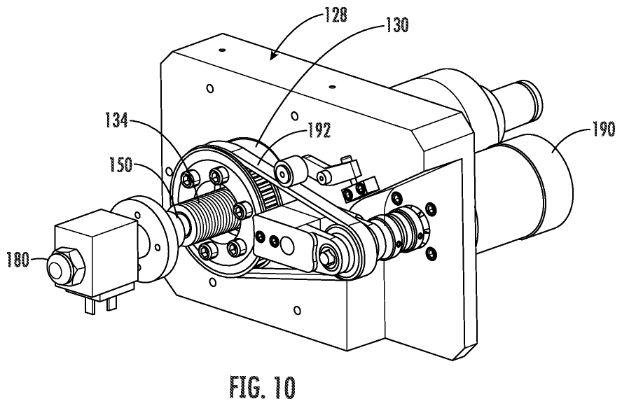

Movement of the piston 170 from a first, fully extended position to a second, retracted position may create a negative pressure so as to draw the one or more vaccines and/or components for making the vaccine or vaccines in through the receiving port 152. Movement of the piston 170 (e.g., release of the piston 170) from the second, retracted position to the first, fully extended position can cause the one or more vaccines and/or components for making the vaccine to be expelled via the nozzle 180. In certain variations, movement of the piston 170 may be controlled by drive motor 190 coupled and supported by the support structure 128. For example, as best illustrated in FIG. 10, the drive motor 190 may be configured to move (e.g., rotate) a belt 192 that is configured to move (e.g., rotate) the ball screw 134. The drive motor 190 may be powered by a battery (not shown) and controlled using a control system. For example, in various aspects, the control system may be configured to initiated movement of the drive motor 190 to cause the piston 170 to be varied by variable degrees, for example using a rotary encounter, allowing the volume of the vaccine injected to be varied.

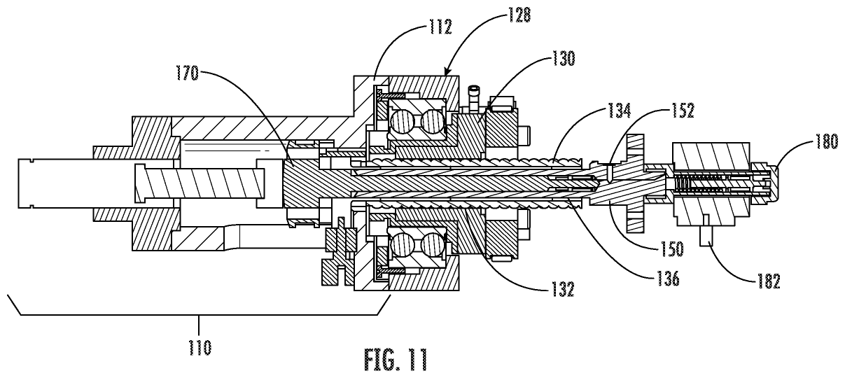

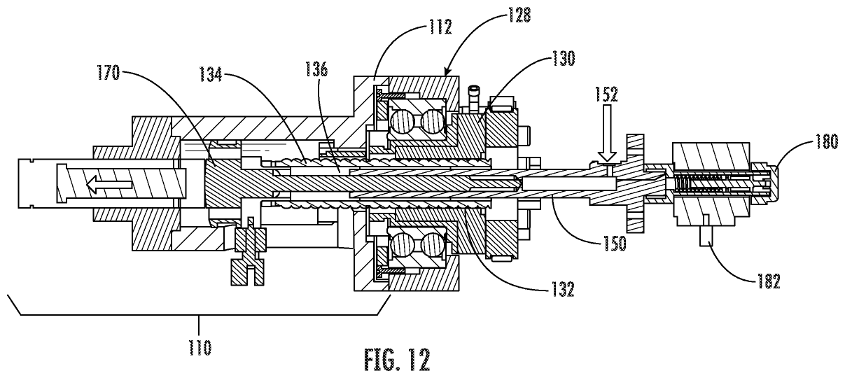

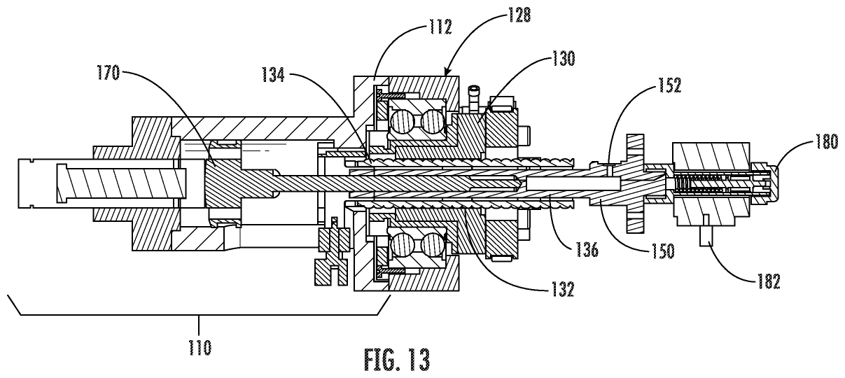

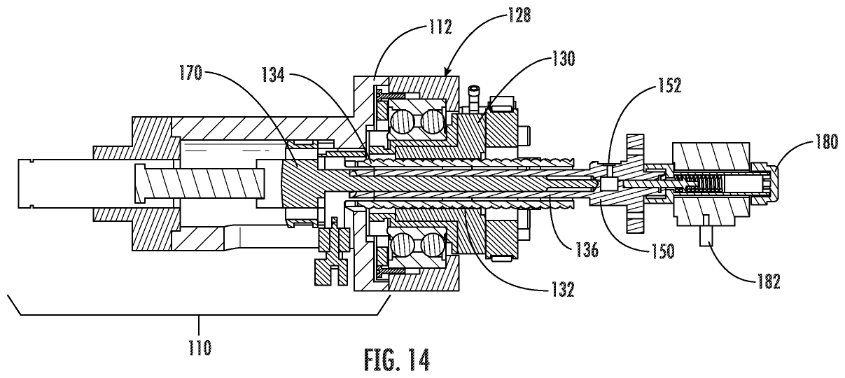

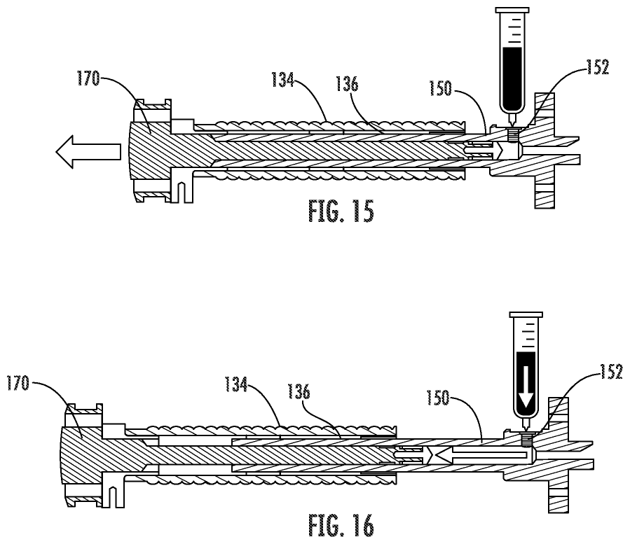

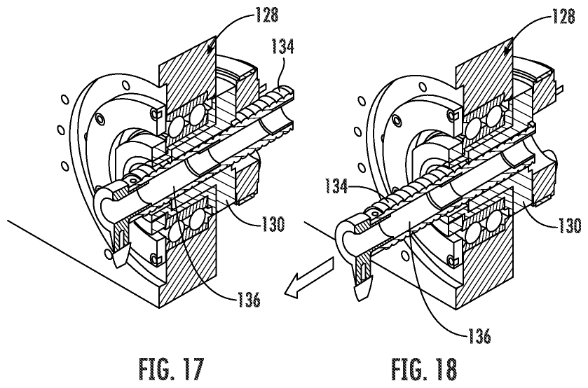

FIGS. 11-14 are partial cross-sectional views of the needle-free injector 100 illustrating the movement of the piston 170 for drawing in and injecting the vaccine. For example, FIG. 11 (like FIGS. 1 and 2) illustrates a resting or starting position, where the gas spring 110, and more specifically, the piston 170 is in a first, fully extended. By way of comparison, FIG. 12 illustrates the piston 170 as it moves from the first, fully extended position to the second, retracted position. As illustrated, the negative pressure created by the movement of the piston 170 from the first position to the second position allows the one or more vaccines and/or components for making the vaccine or vaccines to enter through receiving port 152. FIG. 13 illustrates a loaded state of the needle-free injector 100, where the one or more vaccines and/or components for making the vaccine or vaccines have been pulled into the vaccine dosing chamber 150 and are ready to be administered to the subject. FIG. 14 illustrates the release (or administration) of the one or more vaccines and/or components for making the vaccine or vaccines upon the movement of the one or more valves (or triggers) 182 and opening of the nozzle 180. By way of further illustration, FIGS. 15 and 16 are partial cross-sectional views illustrating the piston 170 as it moves from the first, fully extended position to the second, retracted position, and FIGS. 17 and 18 are additional partial cross-sectional views illustrating the ball screw 134 as it moves from the first, fully extended position to the second, retracted position.

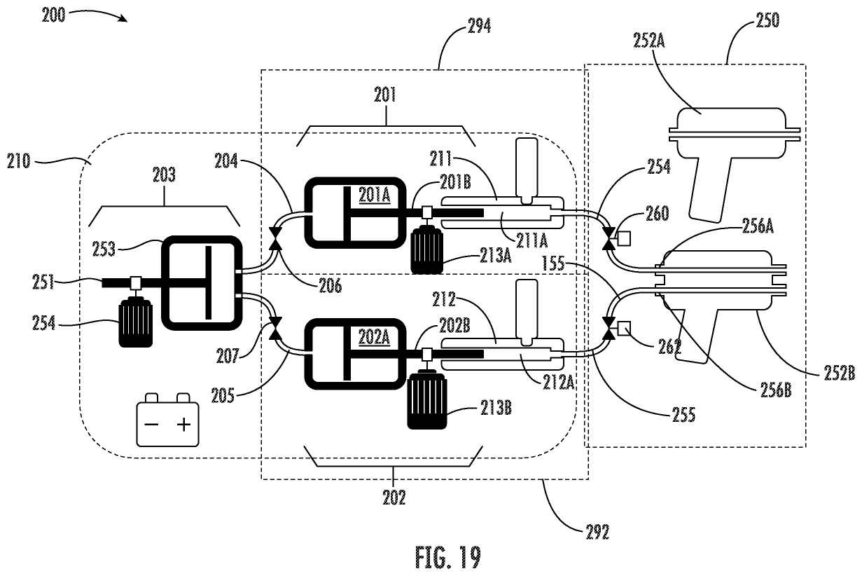

FIG. 19 is a schematic illustration of another example gas-actuated needle-free injector 200 having a first portion (or part) 210 and a second portion (or part) 250. The first portion 210 may be distinct form the second portion 250 in that the first portion 210 is a transportable or wearable component and the second portion 250 is a handheld administration device (e.g., a single nozzle 252A or a double nozzle 252B). The first portion 210 may include one or more gas-actuated components 292, 294 that have configurations similar to the gas-actuated needle-free injectors 100 illustrated in FIGS. 1-18.

As illustrated in FIG. 19, the first portion 210 may include one or more gas-powered, high-pressure vaccine delivery units (e.g., gas springs 201, 202, which defines first sub-parts or portions) in communication with a pressure adjustment cylinder 203 (which defines a second sub-part or portion) that allows the pressure in the gas springs 201, 202 to be adjusted. For example, in certain variations, the first portion 210 may include first and second gas-powered, needle-free injector springs 201, 202 disposed downstream of the pressure adjustment cylinder 203. As illustrated, the first gas-powered, needle-free injector spring 201 may be disposed parallel with the second gas-powered needle-free injector spring 202. The one or more gas-powered, needle-free injector springs 201, 202 may be connected to the pressure adjustment cylinder 203 using one or more connecting hoses or tubes 204, 205. For example, as illustrated, a first connecting hose 204 may connect the first gas-powered, needle-free injector spring 201 to the pressure adjustment cylinder 203, and a second connector hose 205 may connect the second gas-powered, needle-free injector spring 202 to the pressure adjustment cylinder 203.

In certain variations, the one or more connecting hoses 204, 205 may include one or more gate valves 206, 207. For example, as illustrated, the first connecting hose 204 may include a first gate valve 206, and the second connecting hose 205 may include a second gate valve 207. The first gate valve 206 may be positioned at any point along the first connecting hose 204. As illustrated, the first gate valve 206 may be located at a position, for example only, that is about halfway between the pressure adjustment cylinder 203 and the first gas-powered, needle-free injector spring 201. Similarly, the second gate valve 207 may be positioned at any point along the second connecting hose 205. As illustrated, the second gate valve 206 may be located at a position, for example only, that is about halfway between the pressure adjustment cylinder 203 and the second gas-powered, needle-free injector spring 202. The first gate valve 206 may be positioned independently of the second gate valve 207. In each instance, the one or more gate valves 206, 207 may be closed at certain times, for example during a firing stroke of the pressure adjustment cylinder 203, so as to limit or prevent unnecessary gas flow.

The first portion 210 may also include one or move vaccine dosing chambers 211, 212 (defining third sub-parts or portions). For example, a first vaccine dosing chamber 211 may be in communication with the first gas-powered, needle-free injector spring 201, and a second vaccine dosing chamber 212 may be in communication with a second gas-powered, needle-free injector spring 202. As illustrated, each of the gas-powered, needle-free injector springs 201, 202 includes a volume receiving portion 201A, 202A and a plunger or piston 201B, 202B that moves relative the volume receiving portion 201A, 202A. The pistons 201B, 202B may be configured to move between first position and second position. In certain variations, as discussed above, motors 213A, 213B may be in communication with the pistons 201B, 202B to aid the movement of the pistons 201B, 202B from a first, extended position to a second, retracted position. As illustrated, a portion of the piston 201B, 202B extends into a receiving cavity 211A, 212A of one of the one or more vaccine dosing chambers 211, 212 and movement of the piston 201B, 202B may depend upon a pressure in the volume receiving portion 201A, 202A and/or a pressure applied to the piston 201B, 202B. In each variation, the second and third sub-parts may be separately configured to permit greater manufacturing flexibility.

The first portion 210 may be in connection with the second portion 250 via one or more connecting tubes or hoses 254, 255. For example, as illustrated, a third connecting tube 254 may connect the first vaccine dosing chamber 211 and a first entry port 256A of the double nozzle 252B, and a fourth connecting tube 255 may connect the second vaccine dosing chamber 212 and a second entry port 256B of the double nozzle 252B. The one or more connecting hoses 254, 255 may include one or more gate valves 260, 262. For example, as illustrated, the third connecting hose 254 may include a third gate valve 260, and the fourth connecting hose 255 may include a second gate valve 262. The third gate valve 260 may be positioned at any point along the third connecting hose 254. As illustrated, the third gate valve 260 may be located at a position that is, for example only, about halfway between the first vaccine dosing chamber 211 and the first entry port 256A of the double nozzle 252B. Similarly, the fourth gate valve 262 may be positioned at any point along the fourth connecting hose 255. According to some aspects, the gate valves 260, 262 may be positioned inside the double nozzle device 252B so as to locate them close to the nozzles 180.

As illustrated, the fourth gate valve 262 may be located at a position that is, for example only, about halfway between the second vaccine dosing chamber 212 and the second entry port 256B of the double nozzle 252B. The third gate valve 260 may be positioned independently of the fourth gate valve 262. According to some aspects, the connecting hoses 204, 205 (used for air flow) may have comparatively small internal diameters with respect to connecting hoses 254, 255 (used for liquid flow; e.g., vaccine) because pressure is not regulated during a firing stroke of the actuated gas spring 203. For example, in certain variations, the connecting hoses 254, 255 may each have average internal diameters greater than or equal to about 1 millimeters to less than or equal to about 3 millimeters, and in certain aspects, optionally about 2 millimeters. In some instances, the connecting hoses 204, 205 may have the same or larger internal diameter as compared to the connecting hoses 254, 255.

Like the first and second gas-powered, needle-free injector springs 201, 202, the actuated gas spring 203 may include a plunger or piston 251 that moves relative to a volume receiving portion 253. For example, the piston 251 may be configured to move between a first position and a second position. In certain variations, as discussed above, a motor 254 may be in communication with the piston 251 to aid the movement of the piston from a first, extended position to a second, retracted position. As further discussed below, smaller movements of the actuated gas spring 203 may be used to compensate for temperature drifts. For example, in certain variations, the actuated gas spring 203 may be used in combination with one or more pressure sensors (not shown) to ensure that the firing force of the gas-actuated needle-free injector 200 is maintained as temperatures drift. Conversely, larger movements of the actuated gas spring 203 may be used to vary overall pressures, allowing the gas-actuated needle-free injector 200 to be used for different injection depths.

In some instances, it may be desirable to deliver small injection doses (e.g., microdoses) needle-free to a subject. It has been discovered by the inventors, however, that existing needle-free systems are unable to perform such small injection doses accurately due to pressure transients during an initial injection period. That is, such systems become large compared to the transit time of pressure waves, resulting in pressure oscillations that take some time to decay. It has been determined that the cause of such oscillations is due to rigid tubes or hoses, typically mounted in a handpiece or nozzle from which the fluid is delivered, and more particularly at a junction of a rigid tube/hose with a flexible hose (e.g., a hydraulic hose). In this regard, pressure waves in a rigid tube/hose travel at a faster speed than pressure waves in a flexible hydraulic hose. Where a rigid hose and a flexible hose join, there is an impedance mismatch for wave propagation, resulting in reflections and standing waves.

Accordingly, according to some aspects of the present disclosure, the flexible (e.g., elastic) hose 189, such as a flexible hydraulic hose, is in fluid communication with a fluid reservoir and connected within close proximity of the injection orifice 302 of the nozzle 180. According to some aspects the flexible hose 189 may be connected less than about 10 cm from the injection orifice 302. In some instances, the flexible hose 189 may be connected less than about 2 cm from the injection orifice 302. By connecting the flexible hose 189 close to the injection orifice 302, the timescale of oscillations is compressed, so also the initial disturbed flow timescale is also compressed. Such systems may be used to deliver less than 0.5 ml dose injections accurately and with high injected fraction.

According to some aspects, a damping element, such as a viscoelastic material may be incorporated near the nozzle to help dampen any high frequency oscillations. In some instances, an o-ring seal may be used, but in order to provide good dampening function, a non-sealing viscoelastic component may be used, such as an o-ring that is not functioning as a seal, but instead as a volumetric compression dampener.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A gas-actuated needle-free injector comprising:
a gas spring in communication with a piston;
a vaccine dosing chamber configured to receive one or more vaccines or components thereof; and
a ball screw coaxial with the gas spring and the vaccine dosing chamber and configured to receive at least portion of the piston, the ball screw being configured to move the piston between a first position and a second position.

2. The gas-actuated needle-free injector of claim 1, wherein the gas-actuated needle-free injector further comprises:
a first nozzle configured to be contacted to a subject and in fluid communication with the vaccine dosing chamber, the first nozzle comprising a first single injection orifice and configured to produce a single, coherent jet comprising the one or more vaccines.

3. The gas-actuated needle-free injector of claim 2, wherein the gas-actuated needle-free injector comprises a first portion and a second portion, the first portion comprising the gas spring, vaccine dosing chamber, and the ball screw, and the second portion comprising the first nozzle.

4. The gas-actuated needle-free injector of claim 3, wherein the first portion is a wearable component and the second portion is a handheld administration device.

5. The gas-actuated needle-free injector of claim 4, wherein the first portion and the second portion are connected by one or more connecting tubes.

6. The gas-actuated needle-free injector of claim 2, wherein the gas spring is a first gas spring, the vaccine dosing chamber is a first vaccine dosing chamber, and the ball screw is a first ball screw, and the gas-actuated needle-free injector, and the gas-actuated needle-free injector further comprises a second gas spring, a second vaccine dosing chamber, and a second ball screw, wherein the first gas spring, the first vaccine dosing chamber, and the first ball screw define a first assembly and the first assembly is in communication with the first single injection orifice, and the second gas spring, the second vaccine dosing chamber, and the second ball screw define a second assembly.

7. The gas-actuated needle-free injector of claim 6, further comprising a second nozzle configured to be contacted to a subject and in fluid communication with the second vaccine dosing chamber, the second nozzle comprising a second single injection orifice and configured to produce a single, coherent jet comprising one or more vaccines, wherein the second assembly is in communication with the second single injection orifice.

8. The gas-actuated needle-free injector of claim 7, further comprising a first connecting tube comprising one or more first valves that connects the first vaccine dosing chamber and the first single injection orifice, and a second connecting tube comprising one or more second valves that connects the second vaccine dosing chamber and the second single injection orifice.

9. The gas-actuated needle-free injector of claim 8, wherein the first valves and the second valves are disposed within a handheld administration device.

10. The gas-actuated needle-free injector of claim 8, wherein the first and second valves are electrically actuated.

11. The gas-actuated needle-free injector of claim 7, wherein the gas-actuated needle-free injector further comprises a pressure adjustment cylinder that is configured to adjust a first pressure of the first gas spring and a second pressure of the second gas spring.

12. The gas-actuated needle-free injector of claim 11, wherein the gas-actuated needle-free injector further comprises a first tube a first connecting tube comprising one or more first valves that connects the pressure adjustment cylinder and the first gas spring, and a second connecting tube comprising one or more second valves that connects the pressure adjustment cylinder and the second gas spring.

13. The gas-actuated needle-free injector of claim 1, wherein the gas-actuated needle-free injector further comprises a pressure adjustment cylinder that is configured to adjust a pressure of the gas spring.

14. The gas-actuated needle-free injector of claim 13, wherein the gas-actuated needle-free injector further comprises a connecting tube comprising one or more valves that connects the pressure adjustment cylinder and the gas spring.

15. A method of delivering a fluid, the method comprising:
receiving a fluid into a dosing chamber of a gas-actuated needle free-injector, the gas-actuated needle free-injector having a gas spring in communication with a piston, and the gas-actuated needle free-injector further having a ball screw coaxial with the gas spring and the dosing chamber and configured to receive at least portion of the piston, the ball screw being configured to move the piston between a first position and a second position; and
actuating the gas spring so as to discharge the fluid from the dosing chamber.

16. A needle-free injection device, comprising:
a nozzle defining an injection orifice; and
a flexible hose connected to a fluid reservoir, wherein the flexible hose is connected to the nozzle within close proximity of the injection orifice.

17. The needle-free injection device of claim 16, wherein the flexible hose is connected to the nozzle within less than about 10 cm of the injection orifice.

18. The needle-free injection device of claim 16, wherein the flexible hose is connected to the nozzle within less than about 2 cm of the injection orifice.

19. The needle-free injection device of claim 16, wherein the flexible hose is an elastic hydraulic flexible hose.

20. The needle-free injection device of claim 16, further comprising means for moving a fluid from the fluid reservoir to the nozzle for dispensing the fluid from the injection orifice.
